(19) United States
(12) Patent Application Publication
(10) Pub. No.: US 2009/0054811 A1
(43) Pub. Date: Feb. 26, 2009

(54) Method and apparatus for analyte measurement test time

(76) Inventor: Dirk Boecker, Palo Alto, CA (US)

Correspondence Address: HELLER EHRMAN LLP, 4350 La Jolla Village Drive, 7th Floor, San Diego, CA, 92122, US

(21) Appl. No.: 11/813,014

(22) PCT Filed: Dec. 30, 2005
(86) PCT No.: PCT/US2005/047480

§ 371 Date: Apr. 25, 2008

Related U.S. Application Data
(60) Provisional application No. 60/640,879, filed on Dec. 30, 2004.

(51) Int. Cl. A61B 5/151

(52) U.S. Cl. 600/583

(57) ABSTRACT

A method is provided for the analyte measurement by a user using an analyte measurement device. A penetrating member and unused analyte detecting member of the analyte measurement device are presented into an active position. The penetrating member is fired to prick the skin and bring a fluid sample to the analyte detecting member. The analyte level is measured. These three steps occur in less than 10 seconds.

### BACKGROUND OF THE INVENTION

1. Technical Field

The technical field relates to analyte measurement, and more specifically, the amount of time it takes to complete an analyte measurement.

2. Background Art

Lancing devices are known in the medical health-care products industry for piercing the skin to produce blood for analysis. Typically, a drop of blood for this type of analysis is obtained by making a small incision in the fingertip, creating a small wound, which generates a small blood droplet on the surface of the skin.

Early methods of lancing included piercing or slicing the skin with a needle or razor. Current methods utilize lancing devices that contain a multitude of spring, cam and mass actuators to drive the lancet. These include cantilever springs, diaphragms, coil springs, as well as gravity plumbs used to drive the lancet. The device may be held against the skin and mechanically triggered to ballistically launch the lancet. Unfortunately, the pain associated with each lancing event using known technology discourages patients from testing. In addition to vibratory stimulation of the skin as the driver impacts the end of a launcher stop, known spring based devices have the possibility of firing lancets that harmonically oscillate against the patient tissue, causing multiple strikes due to recoil. This recoil and multiple strikes of the lancet is one major impediment to patient compliance with a structured glucose monitoring regime.

Success rate generally encompasses the probability of producing a blood sample with one lancing action, which is sufficient in volume to perform the desired analytical test. The blood may appear spontaneously at the surface of the skin, or may be “milked” from the wound. Milking generally involves pressing the side of the digit, or in proximity of the wound to express the blood to the surface. In traditional methods, the blood droplet produced by the lancing action must reach the surface of the skin to be viable for testing.

When using existing methods, blood often flows from the cut blood vessels but is then trapped below the surface of the skin, forming a hematoma. In other instances, a wound is created, but no blood flows from the wound. In either case, the lancing process cannot be combined with the sample acquisition and testing step. Spontaneous blood droplet generation with current mechanical launching system varies between launcher types but on average it is about 50% of lancet strikes, which would be spontaneous. Otherwise milking is required to yield blood. Mechanical launchers are unlikely to provide the means for integrated sample acquisition and testing if one out of every two strikes does not yield a spontaneous blood sample.

Many diabetic patients (insulin dependent) are required to self-test for blood glucose levels five to six times daily. The large number of steps required in traditional methods of glucose testing ranging from lancing, to milking of blood, applying blood to the test strip, and getting the measurements from the test strip discourages many diabetic patients from testing their blood glucose levels as often as recommended. Tight control of plasma glucose through frequent testing is therefore mandatory for disease management. The pain associated with each lancing event further discourages patients from testing. Additionally, the wound channel left on the patient by known systems may also be of a size that discourages those who are active with their hands or who are worried about healing of those wound channels from testing their glucose levels.

Another problem frequently encountered by patients who must use lancing equipment to obtain and analyze blood samples is the amount of manual dexterity and hand-eye coordination required to properly operate the lancing and sample testing equipment due to retinopathies and neuropathies particularly, severe in elderly diabetic patients. For those patients, operating existing lancet and sample testing equipment can be a challenge. Once a blood droplet is created, that droplet must then be guided into a receiving channel of a small test strip or the like. If the sample placement on the strip is unsuccessful, repetition of the entire procedure including re-lancing the skin to obtain a new blood droplet is necessary.

Early methods of using test strips required a relatively substantial volume of blood to obtain an accurate glucose measurement. This large blood requirement made the monitoring experience a painful one for the user since the user may need to lance deeper than comfortable to obtain sufficient blood generation. Alternatively, if insufficient blood is spontaneously generated, the user may need to “milk” the wound to squeeze enough blood to the skin surface. Neither method is desirable as they take additional user effort and may be painful. The discomfort and inconvenience associated with such lancing events may deter a user from testing their blood glucose levels in a rigorous manner sufficient to control their diabetes.

A further impediment to patient compliance is the amount of time it takes for a user to obtain an analyte measurement using known devices. There are typically several devices in separate packaging that are typically brought together to perform the testing. These multiple devices such as test strips, lancets, a meter, and/or a lancet launcher all increase the complexity and burden on a user.

There is a need to provide methods for reducing the total test time for a user to complete an analyte measurements using analyte measurement devices.

### SUMMARY OF THE INVENTION

Accordingly, an object of the present invention is to provide a method for improving analyte measurement test time and convenience.

Another object of the present invention is to provide a method for improving glucose measurement test time and convenience.

Yet another embodiment of the present invention is to provide a method for measuring an analyte with an analyte measurement device in less than 10 seconds.

A further object of the present invention is to provide a method for measuring analyte with an analyte measurement device that has penetrating members, that is quick and does not require the user to directly handle the penetrating members

Another object of the present invention is to provide a method for measuring analyte with an analyte measurement device that has penetrating members, that is quick and does not require the user to remove and dispose of the penetrating members from the analyte measurement device.

Yet another object of the present invention is to provide a method for measuring analyte with an analyte measurement device that has penetrating members, that is quick and where the analyte measure device is ready for the next lancing event without having to dispose of the used penetrating member or a used analyte detecting member.

These and other objects of the present invention are achieved in, a method of analyte measurement by a user using an analyte measurement device. A penetrating member and unused analyte detecting member of the analyte measurement device are presented into an active position. The penetrating member is fired to prick the skin and bring a fluid sample to the analyte detecting member. The analyte level is measured. These three steps occur in less than 10 seconds.

In another embodiment of the present invention, a method of analyte measurement by a user uses an analyte measurement device. A decision is made to test. A penetrating member and unused analyte detecting member of the analyte measurement device is presented into an active position. The penetrating member is fired to prick the skin and bring a fluid sample to the analyte detecting member. The analyte level is measured. These four steps occur in no more than 1 minute.

In another embodiment of the present invention, a method of analyte measurement is performed with an analyte measurement device. A penetrating member and unused analyte detecting member of the analyte measurement device is presented into an active position by rotating a disposable device to align in an active position. Seals covering the penetrating member and analyte detecting member are removed. The penetrating member is fired to prick the skin using a driver to advance and retract from the skin to create a wound from which body fluid expresses. A fluid sample is brought to the analyte detecting member by providing a sample capture structure positioned to contact body fluid expressed from the wound. The analyte levels are measured. These steps occur in no more than 10 seconds.

### BRIEF DESCRIPTION OF THE DRAWINGS

FIG. 1 is a flow chart illustrating one method of the present invention.

FIG. 2 illustrates an embodiment of a penetrating member driver that can used with the methods of the present invention.

FIGS. 3(a) and 3(b) illustrate embodiments of displacement and velocity profiles, respectively, of a harmonic spring/mass powered driver that can be used with the methods of the present invention.

FIG. 3(c) illustrates an embodiment of a controlled displacement profile that can be utilized with the methods of the present invention.

FIG. 3(d) illustrates an embodiment of a the controlled velocity profile that can be used with the methods of the present invention.

FIG. 4 illustrates a feedback loop and a processor that can be used with the methods of the present invention.

FIG. 5 illustrates a tissue penetration device, more specifically, a lancing device and a controllable driver coupled to a tissue penetration element, that can be used with the methods of the present invention.

FIG. 6 illustrates the lancing device of FIG. 5 in more detail.

FIG. 7 is a partial sectional view of a disposable device that can be utilized with the methods of the present invention.

FIG. 8 is a full sectional view of the FIG. 7 disposable device.

### DESCRIPTION OF THE SPECIFIC EMBODIMENTS

The present invention provides a solution for body fluid sampling. Specifically, some embodiments of the present invention provide improved devices and methods for storing a sampling device. The invention may use a high density penetrating member design. It may use penetrating members of smaller size, such as but not limited to diameter or length, than those of conventional penetrating members known in the art. The device may be used for multiple lancing events without having to remove a disposable from the device. The invention may provide improved sensing capabilities. At least some of these and other objectives described herein will be met by embodiments of the present invention.

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

In one embodiment of the present invention, a method is provided for doing an analyte measurement by a user using an analyte measurement device in three steps. In a first step, a penetrating member and unused analyte detecting member of the analyte measurement device are presented into an active position. In a second step, the penetrating member is fired to prick the skin and bring a fluid sample to the analyte detecting member. In a third step, the analyte level is measured. In one embodiment, these three steps occur in less than 10 seconds. In another embodiment, these steps occur in less than 7 seconds. The analyte level can be displayed to the use, and a value of the analyte level can be stored in or out of the analyte measurement device.

These three steps can be performed without the user directly handling the penetrating member to obtain a fresh penetrating member or load the penetrating member, and/or without the user coding the analyte measurement device. Blood is applied to an analyte detection member during lancing. Application of blood to an analyte detection member during lancing occurs without removal and disposal of penetrating members from the analyte measurement device. The three steps can be performed without a separate step of apply blood to a analyte detection member after lancing. The second step can be performed without milking a wound. The second step can be performed using at least one of a penetrating member driver selected from, spring based, electro-mechanical based, magnetic driver based, and nanomuscle based.

The second step can be performed with controlled velocity and depth of penetration, as more fully described hereafter. The analyte measurement device can be returned to a storage condition without having to dispose of a used penetrating member or used analyte detecting members. The analyte measurement device is ready for the next lancing event without having to dispose of the used penetrating member or the used analyte detecting member. In one embodiment, a time from pressing an on button of the analyte measurement device to lancing and measuring the analyte level is no more than 10 seconds.

From the moment the user thinks that it is time to do an analyte measurement (and begins the test process, reaches for the analyte measurement device, or initiates movement to begin the testing) to the time that a reading appears, in one embodiment, the present invention desires to be 10 seconds or less.

The test time breaks down into smaller pieces. The user will desire to do a test and then grab their measurement kit. In some embodiments, the user will take some action to turn on the analyte measurement device and take some action to prepare it. The user would hold the analyte measurement device to their skin and then first with some action by the user. Thus so far, the user will turn on the analyte measurement device, prepare it, and fire it. This may be combined into one. The time it takes is about 2 seconds to fire, 2 process to interact, and 4 seconds to get your readings.

A user right now will take about 20 seconds if certain steps are skipped. If the proper steps are taken then it takes a user about a minute. It is unlikely that a user may improve by a second or two if a second person helps. The speed is based on someone with dexterity to do things quickly. In one embodiment, the present invention provides a testing regime that removes much of the user variability and dexterity to testing.

In one embodiment of the present invention, the user does not need to dispose of or handle waste materials after each testing event, the user does not need to put the lancer back in place, the via back in place, or meter back in place. The present invention can offer a single analyte measurement device. The present invention can allow a user to get their reading and the put the analyte measurement device back down to where they had it. Whatever the user needs to do to return the analyte measurement device to their normal state or storage state is the end point of the time measurement.

The present invention removes taking a strip out of a vial, putting a strip into a meter, disposing of the strip, eliminate the need to grab a separate lancing device, eliminate the transfer step from a finger to a test strip.

The starting point for measuring may be when they open the carrying case or grabbing the test strip vial (to begin a test process). This may involve press the button or slide the slider to produce the test strip from the analyte measurement device. The step of physically preparing the strip is removed. Some users will leave the meter in a carrying case.

The present invention is the lower test time and the removal of certain steps. The present invention provides a convenience factor. Even though some steps will be reduced in time, the number of steps to reach a reading is improved. The user may wait less, but there is no reduction in convenience. The absolute time is more of a benefit of reduced steps. Even the automatically dispensing test strip devices still have the step of placing the strip and then removing it when done. There are no elimination of steps.

In the present invention, opening a latch or other trigger on the analyte measurement device may be used to prepare the analyte measurement device to have more device steps performed by fewer user steps. A latch may be opened and this may allow the analyte measurement device to power up and advance for next lancing event.

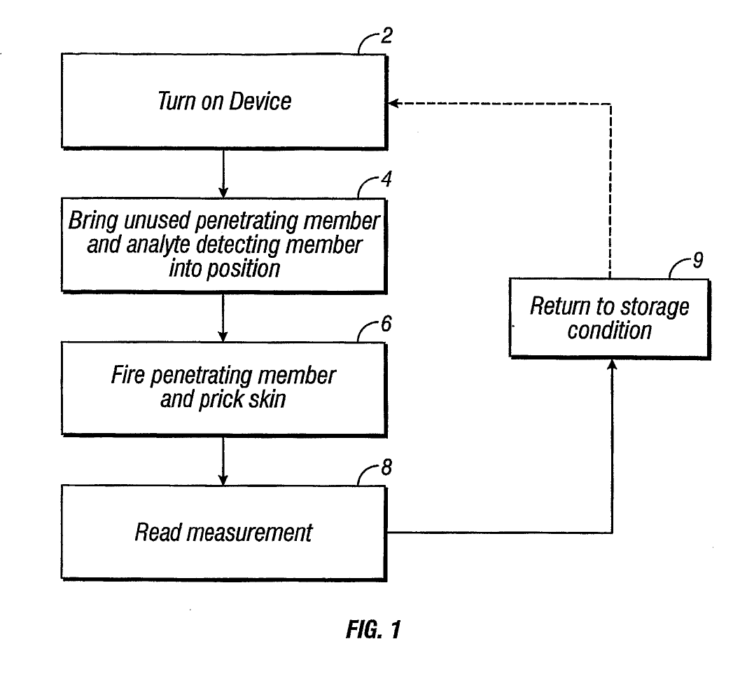

FIG. 1 is a flow chart of one embodiment of a method of the present invention. The analyte measurement device may be turned on at step 2. In some embodiments, the turn on at step 2 also performs the bringing of an unused penetrating member (and analyte detecting member as the case may be) into position. Some embodiments of the present invention has an explicit step 4 for bringing an unused penetrating member and analyte detecting member into position. Step 6 shows that the user may fire the analyte measurement device by a variety of methods including but not limited to pressing a button on the analyte measurement device. The firing will prick the skin and bring a blood sample into the analyte measurement device. The user then waits to see a measurement at step 8. At step 9, the user replaces the analyte measurement device into its storage condition, perhaps in a carrying case or by simply placing it back where the user stores testing devices. As indicated by the phantom line, the user will proceed back to step 2 when time comes for the next lancing event.

The present invention desires to complete the end-to-end testing process in less than 10 seconds. In some embodiments, the testing process is completed in less than 11, 12, 13, 14, 15, 16, 17, 18, 19, or 20 seconds. The present invention provides greater convenience by eliminating certain step but still arrive at the same end result of obtaining an analyte measurement.

It should be understood that one way to view the present invention is the number of steps performed by the user and the number of steps performed by the analyte measurement device. The present invention shifts the number of steps performed by the user and minimizes those steps while increasing the number of steps performed by analyte measurement device. Thus in one embodiment, the user may perform four steps (turn on, activate new penetrating member/analyte detecting member, pick skin, return meter to storage condition), the analyte measurement device will perform additional steps not seen by the user (rotate cartridge to bring new penetrating member in position, obtain sample from skin prick, transfer sample to detecting member, store used penetrating member, store used #analyte testing device. The present invention involves removing some steps completely and shifting many of the steps into the analyte measurement device.

In another embodiment of the present invention, a method of analyte measurement by a user uses an analyte measurement device in four steps. In the first step, a decision is made to test. In the second step, a penetrating member and unused analyte detecting member of the analyte measurement device is presented into an active position. In the third step, the penetrating member is fired to prick the skin and bring a fluid sample to the analyte detecting member. In the fourth step, the analyte level is measured. These four steps occur in no more than 1 minute. In various embodiments, steps one through four occur in more than, 30 seconds, 15 seconds, 10 seconds, and the like.

In another embodiment of the present invention, a method of analyte measurement is performed with an analyte measurement device in four steps. In a first step, a penetrating member and unused analyte detecting member of the analyte measurement device is presented into an active position by rotating a disposable device to align in an active position. Seals covering the penetrating member and analyte detecting member are removed. In a second step, the penetrating member is fired to prick the skin using a driver to advance and retract from the skin to create a wound from which body fluid expresses. In a third step, a fluid sample is brought to the analyte detecting member by providing a sample capture structure positioned to contact body fluid expressed from the wound. In a fourth step, the analyte levels are measured. In one embodiment, these four steps occur in no more than 10 seconds. In various embodiments, these four steps are performed without the user, directly handling the penetrating member to obtain a fresh penetrating member or load the penetrating member, or coding the analyte measurement device.

In one embodiment, the four steps are performed without a separate step of apply blood to a analyte detection member after lancing. In another embodiment, the second and third steps are performed without milking a wound.

The analyte level can be displayed to the use, and a value of the analyte level can be stored in or out of the analyte measurement device. Blood is applied to an analyte detection member during lancing. Application of blood to an analyte detection member during lancing occurs without removal and disposal of penetrating members from the analyte measurement device.

In one embodiment, the second and third steps are performed using at least one of a penetrating member driver selected from, spring based, electromechanical based, magnetic driver based, and nanomuscle based. In another embodiment, the third step is performed with controlled velocity and depth of penetration.

The analyte measurement device can be returned to a storage condition without having to dispose of a used penetrating member or used analyte detecting members. The analyte measurement device is ready for the next lancing event without having to dispose of the used penetrating member or the used analyte detecting member. In one embodiment, a time from pressing an on button of the analyte measurement device to lancing and measuring the analyte level is no more than 10 seconds. In another embodiment, there is no disposal of a used analyte detecting member and a used penetrating member after each lancing step. In another embodiment, the four steps are performed without a disposal or handling of waste step.

The present invention may be used with a variety of different penetrating member drivers. It is contemplated that these penetrating member drivers may be spring based, solenoid based, magnetic driver based, nanomuscle based, or based on any other mechanism useful in moving a penetrating member along a path into tissue. It should be noted that the present invention is not limited by the type of driver used with the penetrating member feed mechanism. One suitable penetrating member driver for use with the present invention is shown in FIG. 1. This is an embodiment of a solenoid type electromagnetic driver that is capable of driving an iron core or slug mounted to the penetrating member assembly using a direct current (DC) power supply. The electromagnetic driver includes a driver coil pack that is divided into three separate coils along the path of the penetrating member, two end coils and a middle coil. Direct current is alternated to the coils to advance and retract the penetrating member. Although the driver coil pack is shown with three coils, any suitable number of coils may be used, for example, 4, 5, 6, 7 or more coils may be used.

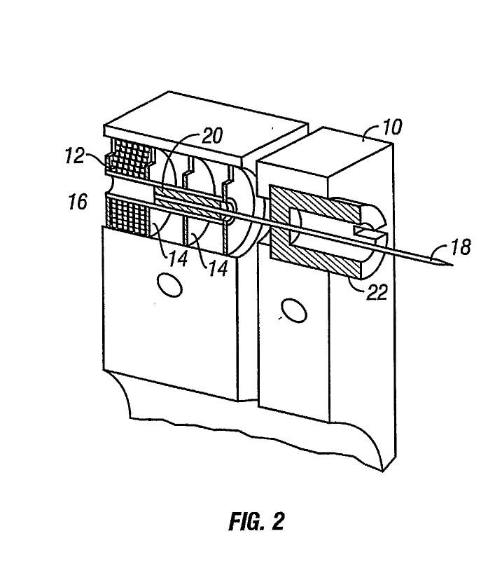

Referring to the embodiment of FIG. 2, a stationary iron housing 10 may contain the driver coil pack with a first coil 12 flanked by iron spacers 14 which concentrate the magnetic flux at the inner diameter creating magnetic poles. The inner insulating housing 16 isolates the penetrating member 18 and iron core 20 from the coils and provides a smooth, low friction guide surface. The penetrating member guide 22 further centers the penetrating member 18 and iron core 20. The penetrating member 18 is protracted and retracted by alternating the current between the first coil 12, the middle coil, and the third coil to attract the iron core 20. Reversing the coil sequence and attracting the core and penetrating member back into the housing retracts the penetrating member. The penetrating member guide 22 also serves as a stop for the iron core 20 mounted to the penetrating member 18.

As discussed above, analyte measurement devices which employ spring or cam driving methods have a symmetrical or nearly symmetrical actuation displacement and velocity profiles on the advancement and retraction of the penetrating member. In most of the available analyte measurement devices, once the launch is initiated, the stored energy determines the velocity profile until the energy is dissipated. Controlling impact, retraction velocity, and dwell time of the penetrating member within the tissue can be useful in order to achieve a high success rate while accommodating variations in skin properties and minimize pain. Advantages can be achieved by taking into account of the fact that tissue dwell time is related to the amount of skin deformation as the penetrating member tries to puncture the surface of the skin and variance in skin deformation from patient to patient based on skin hydration.

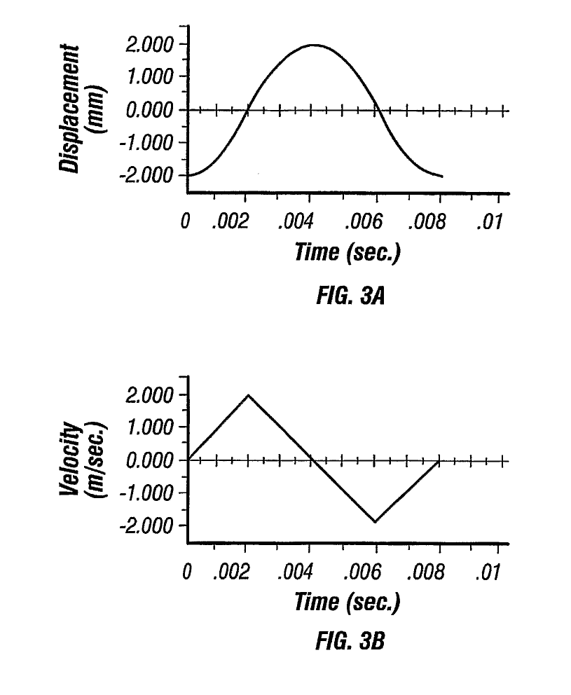

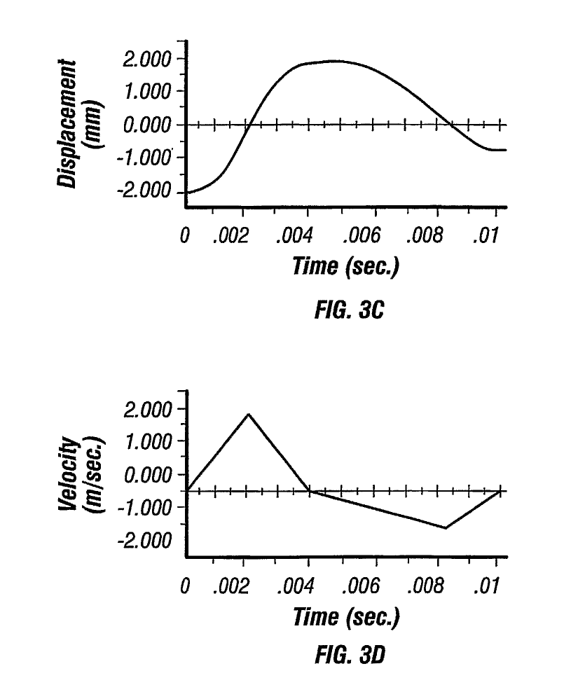

In this embodiment, the ability to control velocity and depth of penetration may be achieved by use of a controllable force driver where feedback is an integral part of driver control. Such drivers can control either metal or polymeric penetrating members or any other type of tissue penetration element. The dynamic control of such a driver is illustrated in FIG. 3(c) which illustrates an embodiment of a controlled displacement profile and FIG. 3(d) which illustrates an embodiment of a the controlled velocity profile. These are compared to FIGS. 3(a) and 3(b), which illustrate embodiments of displacement and velocity profiles, respectively, of a harmonic spring/mass powered driver. Reduced pain can be achieved by using impact velocities of greater than about 2 m/s entry of a tissue penetrating element, such as a lancet, into tissue. Other suitable embodiments of the penetrating member driver are described in commonly assigned, copending U.S. patent application Ser. No. 10/127,395, (Attorney Docket No. 38187-2551) filed Apr. 19, 2002 and previously incorporated herein.

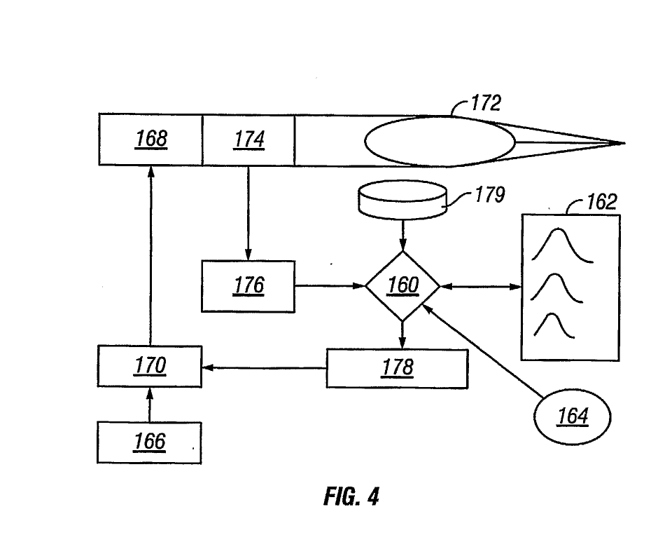

FIG. 4 illustrates the operation of a feedback loop using a processor 60. The processor 60 stores profiles 62 in non-volatile memory. A user inputs information 64 about the desired circumstances or parameters for a lancing event. The processor 60 selects a driver profile 62 from a set of alternative driver profiles that have been preprogrammed in the processor 60 based on typical or desired analyte measurement device performance determined through testing at the factory or as programmed in by the operator. The processor 60 may customize by either scaling or modifying the profile based on additional user input information 64. Once the processor has chosen and customized the profile, the processor 60 is ready to modulate the power from the power supply 66 to the penetrating member driver 68 through an amplifier 70. The processor 60 may measure the location of the penetrating member 72 using a position sensing mechanism 74 through an analog to digital converter 76 linear encoder or other such transducer. Examples of position sensing mechanisms have been described in the embodiments above and may be found in the specification for commonly assigned, copending U.S. patent application Ser. No. 10/127,395, (Attorney Docket No. 38187-2551) filed Apr. 19, 2002 and previously incorporated herein. The processor 60 calculates the movement of the penetrating member by comparing the actual profile of the penetrating member to the predetermined profile. The processor 60 modulates the power to the penetrating member driver 68 through a signal generator 78, which may control the amplifier 70 so that the actual velocity profile of the penetrating member does not exceed the predetermined profile by more than a preset error limit. The error limit is the accuracy in the control of the penetrating member.

After the lancing event, the processor 60 can allow the user to rank the results of the lancing event. The processor 60 stores these results and constructs a database 80 for the individual user. Using the database 79, the processor 60 calculates the profile traits such as degree of painlessness, success rate, and blood volume for various profiles 62 depending on user input information 64 to optimize the profile to the individual user for subsequent lancing cycles. These profile traits depend on the characteristic phases of penetrating member advancement and retraction. The processor 60 uses these calculations to optimize profiles 62 for each user. In addition to user input information 64, an internal clock allows storage in the database 79 of information such as the time of day to generate a time stamp for the lancing event and the time between lancing events to anticipate the user's diurnal needs. The database stores information and statistics for each user and each profile that particular user uses.

In addition to varying the profiles, the processor 60 can be used to calculate the appropriate penetrating member diameter and geometry suitable to realize the blood volume required by the user. For example, if the user requires about 1-5 microliter volume of blood, the processor 60 may select a 200 micron diameter penetrating member to achieve these results. For each class of lancet, both diameter and lancet tip geometry, is stored in the processor 60 to correspond with upper and lower limits of attainable blood volume based on the predetermined displacement and velocity profiles.

The analyte measurement device is capable of prompting the user for information at the beginning and the end of the lancing event to more adequately suit the user. The goal is to either change to a different profile or modify an existing profile. Once the profile is set, the force driving the penetrating member is varied during advancement and retraction to follow the profile. The method of lancing using the analyte measurement device comprises selecting a profile, lancing according to the selected profile, determining lancing profile traits for each characteristic phase of the lancing cycle, and optimizing profile traits for subsequent lancing events.

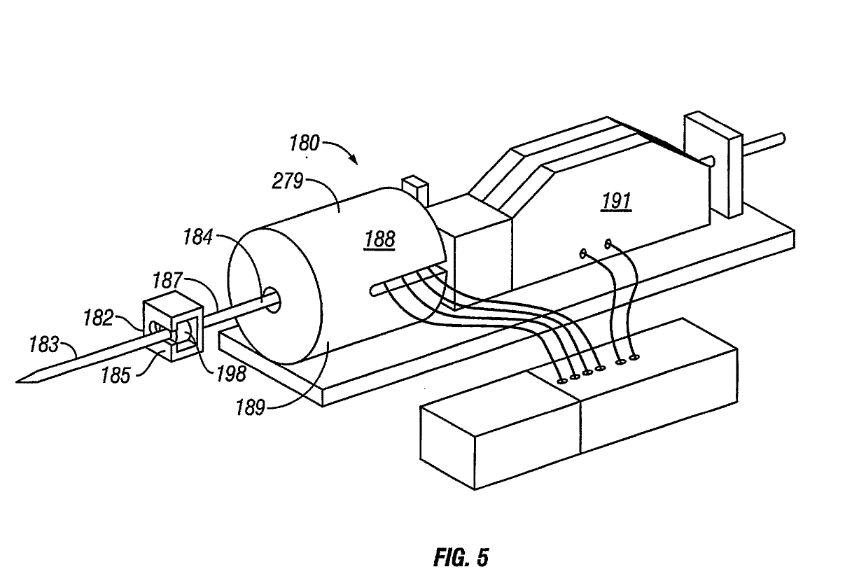

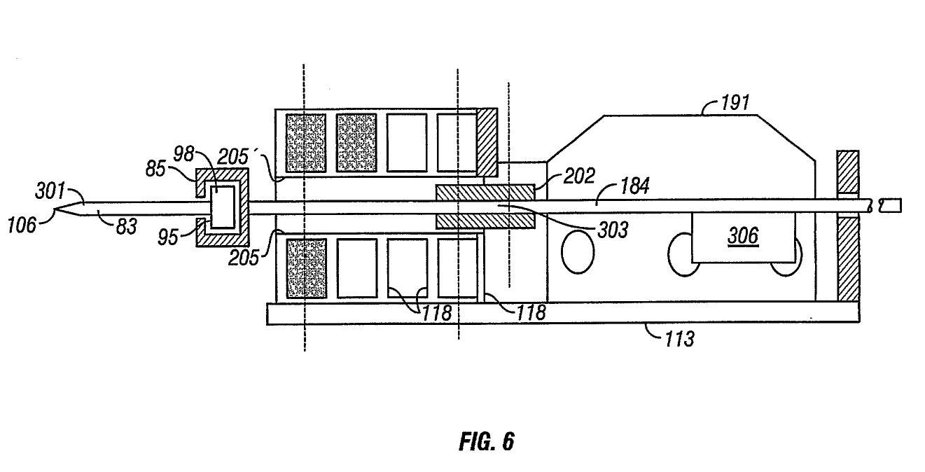

FIG. 5 illustrates an embodiment of an analyte measurement device, more specifically, a lancing device 80 that includes a controllable driver 79 coupled to a tissue penetration element. The lancing device 80 has a proximal end 81 and a distal end 82. At the distal end 82 is the tissue penetration element in the form of a penetrating member 83, which is coupled to an elongate coupler shaft 84 by a drive coupler 85. The elongate coupler shaft 84 has a proximal end 86 and a distal end 87. A driver coil pack 88 is disposed about the elongate coupler shaft 84 proximal of the penetrating member 83. A position sensor 91 is disposed about a proximal portion 92 (FIG. 6) of the elongate coupler shaft 84 and an electrical conductor 94 electrically couples a processor 93 to the position sensor 91. The elongate coupler shaft 84 driven by the driver coil pack 88 controlled by the position sensor 91 and processor 93 form the controllable driver, specifically, a controllable electromagnetic driver.

Referring to FIG. 6, the lancing device 80 can be seen in more detail, in partial longitudinal section. The penetrating member 83 has a proximal end 95 and a distal end 96 with a sharpened point at the distal end 96 of the penetrating member 83 and a drive head 98 disposed at the proximal end 95 of the penetrating member 83. A penetrating member shaft 101 is disposed between the drive head 98 and the sharpened point 97. The penetrating member shaft 101 may be comprised of stainless steel, or any other suitable material or alloy and have a transverse dimension of about 0.1 to about 0.4 mm. The penetrating member shaft may have a length of about 3 mm to about 50 mm, specifically, about 15 mm to about 20 mm. The drive head 98 of the penetrating member 83 is an enlarged portion having a transverse dimension greater than a transverse dimension of the penetrating member shaft 101 distal of the drive head 98. This configuration allows the drive head 98 to be mechanically captured by the drive coupler 85. The drive head 98 may have a transverse dimension of about 0.5 to about 2 mm.

A magnetic member 102 is secured to the elongate coupler shaft 84 proximal of the drive coupler 85 on a distal portion 203 of the elongate coupler shaft 84. The magnetic member 102 is a substantially cylindrical piece of magnetic material having an axial lumen 104 extending the length of the magnetic member 102. The magnetic member 102 has an outer transverse dimension that allows the magnetic member 102 to slide easily within an axial lumen 105 of a low friction, possibly lubricious, polymer guide tube 106 disposed within the driver coil pack 88. The magnetic member 102 may have an outer transverse dimension of about 1.0 to about 5.0 mm, specifically, about 2.3 to about 2.5 mm. The magnetic member 102 may have a length of about 3.0 to about 5.0 mm, specifically, about 4.7 to about 4.9 mm. The magnetic member 102 can be made from a variety of magnetic materials including ferrous metals such as ferrous steel, iron, ferrite, or the like. The magnetic member 102 may be secured to the distal portion 203 of the elongate coupler shaft 84 by a variety of methods including adhesive or epoxy bonding, welding, crimping or any other suitable method.

Proximal of the magnetic member 102, an optical encoder flag 106 is secured to the elongate coupler shaft 84. The optical encoder flag 106 is configured to move within a slot in the position sensor 91. The slot may have separation width of about 1.5 to about 2.0 mm. The optical encoder flag 106 can have a length of about 14 to about 18 mm, a width of about 3 to about 5 mm and a thickness of about 0.04 to about 0.06 mm.

The optical encoder flag 106 interacts with various optical beams generated by LEDs disposed on or in the position sensor 91 in a predetermined manner. The interaction of the optical beams generated by the LEDs of the position sensor 91 generates a signal that indicates the longitudinal position of the optical flag 106 relative to the position sensor 91 with a substantially high degree of resolution. The resolution of the position sensor 91 may be about 200 to about 400 cycles per inch, specifically, about 350 to about 370 cycles per inch. The position sensor 91 may have a speed response time (position/time resolution) of 0 to about 120,000 Hz, where one dark and light stripe of the flag constitutes one Hertz, or cycle per second. The position of the optical encoder flag 206 relative to the magnetic member 102, driver coil pack 88 and position sensor 91 is such that the position sensor 91 can provide precise positional information about the penetrating member 83 over the entire length of the penetrating member's power stroke.

An optical encoder that is suitable for the position sensor 91 is a linear optical incremental encoder, model HEDS 9200, manufactured by Agilent Technologies. The model HEDS 9200 may have a length of about 20 to about 30 mm, a width of about 8 to about 12 mm, and a height of about 9 to about 11 mm. Although the position sensor 91 illustrated is a linear optical incremental encoder, other suitable position sensor embodiments could be used, provided they posses the requisite positional resolution and time response. The HEDS 9200 is a two channel device where the channels are 90 degrees out of phase with each other. This results in a resolution of four times the basic cycle of the flag. These quadrature outputs make it possible for the processor to determine the direction of penetrating member travel. Other suitable position sensors include capacitive encoders, analog reflective sensors, such as the reflective position sensor discussed above, and the like.

A coupler shaft guide 111 is disposed towards the proximal end 81 of the lancing device 80. The guide 111 has a guide lumen 112 disposed in the guide 111 to slidingly accept the proximal portion 92 of the elongate coupler shaft 84. The guide 111 keeps the elongate coupler shaft 84 centered horizontally and vertically in the slot 102, of the position sensor 91.

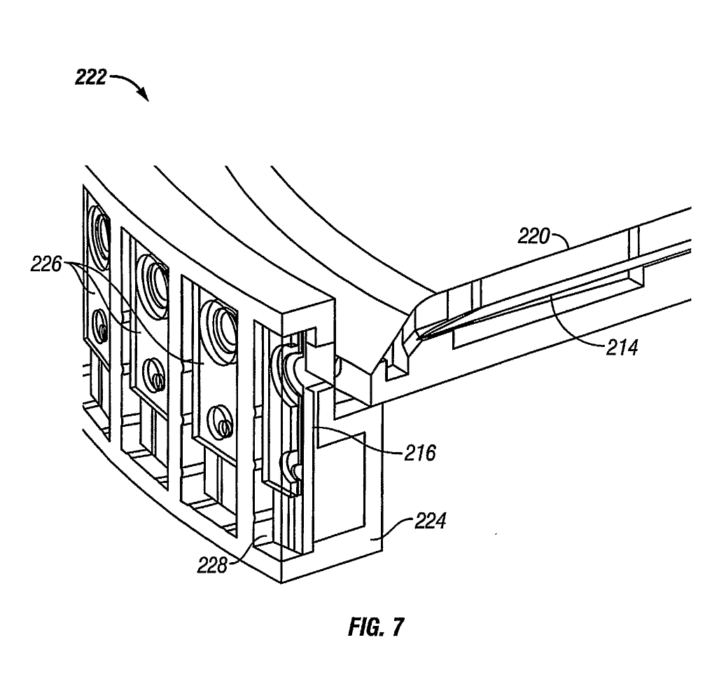

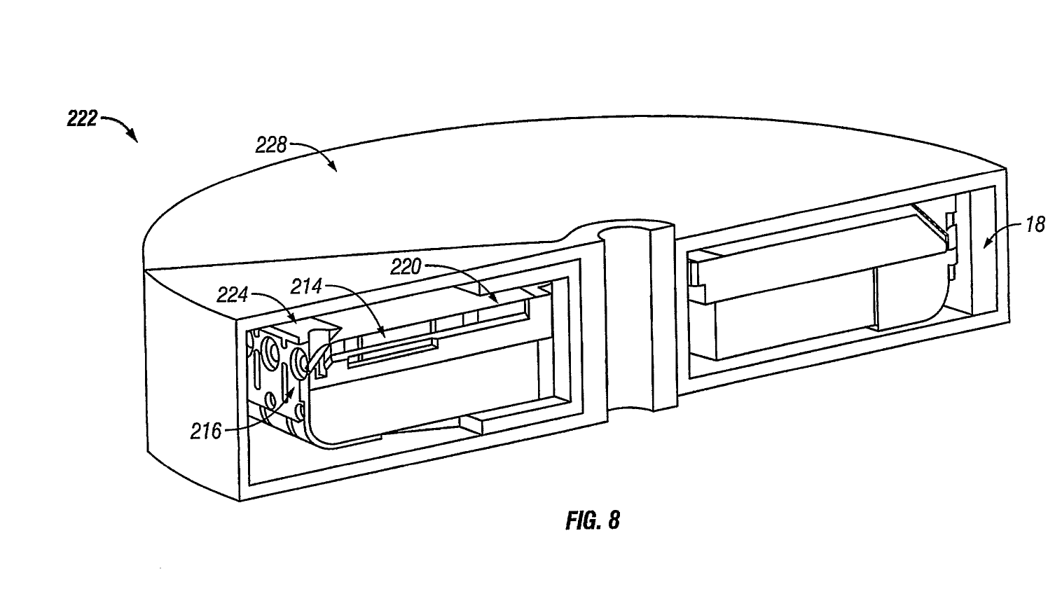

As shown in FIGS. 7 and 8, a plurality of penetrating members 214 can be in a disposable member 222 that is placed in a housing of the analyte measurement device. A plurality of analyte detecting members 216 are also included. Each of an analyte detecting member 16 is coupled to a penetrating member 214. A sterility barrier 220 is configured to provide sterile environments for the plurality of penetrating members 214. The sterility barrier 220 can be made of a variety of materials including but not limited to, a metallic foil or other seal materials and may be of a tensile strength and other quality that may provide a sealed, sterile environment until the sterility barrier 220 is penetrated by a penetrating device 214, providing a preselected or selected amount of force to open the sealed, sterile environment.

The sterility barrier 220 can be a planar material that is adhered to a surface of the disposable device 222. Depending on the orientation of the disposable device 222, the sterility barrier 220 can be on the top surface, side surface, bottom surface, or other positioned surface of the disposable device 222.

The plurality of analyte detecting members 216 can be supported on a scaffolding 224. The scaffolding 224 can be attached to a bottom surface of the disposable device 222. The scaffolding 224 can be made of a material such as, but not limited to, a polymer, a foil, and the like. The scaffolding 224 can hold a plurality of analyte detecting members 216, such as but not limited to, about 10-50, 50-100, or other combinations of analyte detecting members 216. This facilitates the assembly and integration of analyte detecting members 216 with disposable device 222. These analyte detecting members 216 can enable an integrated body fluid sampling system where the penetrating members 214 create a wound tract in a target tissue, which expresses body fluid that flows into the disposable device 222 for analyte detection by at least one of the analyte detecting members 216.

In one embodiment, many analyte detecting members 216 can be printed onto a single scaffolding 224 which is then adhered to the disposable device 222 to facilitate manufacturing and simplify assembly. The analyte detecting members 216 can be electrochemical in nature. The analyte detecting members 216 can further contain enzymes, dyes, or other detectors which react when exposed to the desired analyte. Additionally, the analyte detecting members 216 can comprise of clear optical windows that allow light to pass into the body fluid for analyte analysis. The number, location, and type of analyte detecting member 216 can be varied as desired, based in part on the design of the disposable device 222, number of analytes to be measured, the need for analyte detecting member calibration, and the sensitivity of the analyte detecting members 216. Wicking elements, capillary tube or other devices on the disposable device 222 can be provided to allow body fluid to flow from the disposable device 222 to the analyte detecting members 216 for analysis. In other configurations, the analyte detecting members 216 can be printed, formed; or otherwise located directly in the disposable device 222.

The disposable device 222 can include a plurality of cavities 226. Each penetrating member 214 may be contained in a cavity 226 in the disposable device 222 with its sharpened end facing radially outward and may be in the same plane as that of the disposable device 222. The cavity 226 may be molded, pressed, forged, or otherwise formed in the disposable device 222. Although not limited in this manner, the ends of the cavities 226 may be divided into individual fingers (such as one for each cavity) on the outer periphery of the disposable device 222. The particular shape of each cavity 226 may be designed to suit the size or shape of the penetrating member therein or the amount of space desired for placement of the analyte detecting members 216. For example and not limitation, the cavity 226 may have a V-shaped cross-section, a U-shaped cross-section, C-shaped cross-section, a multi-level cross section or the other cross-sections. The opening through which a penetrating member 214 may exit to penetrate tissue may also have a variety of shapes, such as but not limited to, a circular opening, a square or rectangular opening, a U-shaped opening, a narrow opening that only allows the penetrating member 214 to pass, an opening with more clearance on the sides, a slit, and the like.

The use of the sterility barrier 220 can facilitate the manufacture of disposable device 222. For example, a single sterility barrier 220 can be adhered, attached, or otherwise coupled to the disposable device 222 to seal many of the cavities 226 at one time. A sheet of analyte detecting members 216 can also be adhered, attached, or otherwise coupled to the disposable device 222 to provide many analyte detecting members 216 on or in the disposable device 222 at one time. During manufacturing of one embodiment of the present invention, the disposable device 222 can be loaded with penetrating members 214, sealed with sterility barrier 220 and a temporary layer (not shown) on the bottom where scaffolding 224 would later go, to provide a sealed environment for the penetrating members 214. This assembly with the temporary bottom layer is then taken to be sterilized. After sterilization, the assembly is taken to a clean room (or it can already be in a clear room or equivalent environment) where the temporary bottom layer is removed and the scaffolding 224 with analyte detecting members 216 is coupled to the disposable device 222. This process allows for the sterile assembly of the disposable device 222 with the penetrating members 214 using processes and/or temperatures that can degrade the accuracy or functionality of the analyte detecting members 216 on the scaffolding 224.

In some embodiments, more than one sterility barrier 220 can be used to seal the cavities 226. As examples of some embodiments, multiple layers can be placed over each cavity 226, half or some selected portion of the cavities 226 can be sealed with one layer with the other half or selected portion of the cavities sealed with another sheet or layer, different shaped cavities 226 can use different seal layer, or the like. The sterility barrier 220 can have different physical properties, such as those covering the penetrating members 214 near the end of the disposable device 222 can have a different color such as red to indicate to the user (if visually inspectable) that the user is down to say 10, 5, or other number of penetrating members before the cartridge should be changed out.

After actuation, the penetrating member 214 is returned into the disposable device 222 and is held therein in a manner so that it is not able to be used again. By way of example and not limitation, a used penetrating member 214 may be returned into the disposable member 222 and held by a launcher in position until the next lancing event. At the time of the next lancing, the launcher may disengage the used penetrating member with the disposable device 222 turned or indexed to the next clean penetrating member 214 such that the cavity 226 holding the used penetrating member is positioned so that it is not accessible to the user (i.e. turn away from a penetrating member exit opening). In some embodiments, the tip of a used penetrating member 214 may be driven into a protective stop that hold the penetrating member in place after use. The disposable device 222 is replaceable with a new disposable device 222 once all the penetrating members 214 have been used or at such other time or condition as deemed desirable by the user.

The disposable device 222 can provide sterile environments for penetrating members 214 via the sterility barrier 220, seals, foils, covers, polymeric, or similar materials used to seal the cavities 226 and provide enclosed areas for the penetrating members 214 to rest in. In one embodiment, sterility barrier 220 is applied to one surface of the disposable device 220. Each cavity 226 may be individually sealed in a manner such that the opening of one cavity 226 does not interfere with the sterility in an adjacent or other cavity 226. Additionally, the disposable device 222 can include a moisture barrier 228.

The plurality of penetrating members 214 can be at least partially contained in the cavities 226 of the disposable device 222. The penetrating members 214 are slidably movable to extend outward from the disposable device 222 to penetrate tissue. The cavities 226 can each have a longitudinal opening that provides access to an elongate portion of the penetrating member 214. The sterility barrier 220 can cover the longitudinal openings. The sterility barrier 220 can be configured to be moved so that the elongate portion can be accessed by a gripper without touching the sterility barrier 220.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, the shield or other punch may be adapted for use with other cartridges disclosed herein or in related applications. With any of the above embodiments, the methods time may be measured from when the user touches the carrying case or touches the housing (if the device is not being stored in a carrying case).

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method of analyte measurement by a user using an analyte measurement device, comprising:
(a) presenting a penetrating member and unused analyte detecting member of the analyte measurement device info an active position;
(b) firing the penetrating member to prick the skin and bring a fluid sample to the analyte detecting member;
(c) measuring the analyte level, wherein steps (a) through (c) occur in less than 10 seconds.

2. The method of claim 1, further comprising:
displaying the analyte level to the user

3. The method of claim 1, further comprising:
storing a value of the analyte level.

4. The method of claim 1, wherein steps (a) through (d) occur in less than 7 seconds

5. The method of claim 1, wherein steps (a) through (c) are performed without the user directly handling the penetrating member to obtain a fresh penetrating member or load the penetrating member

6. The method of claim 1, wherein steps (a) through (c) are performed without the user coding the analyte measurement device.

7. The method of claim 1, wherein blood is applied to an analyte detection member during lancing.

8. The method of claim 7, wherein the application of blood to an analyte detection member during lancing occurs without removal and disposal of penetrating members from the analyte measurement device.

9. The method of claim 1, wherein steps (a) through (c) are performed without a separate step of apply blood to a analyte detection member after lancing.

10. The method of claim 1, wherein step (b) is performed without milking a wound.

11. The method of claim 1, wherein step (b) is performed using at least one of a penetrating member driver selected from, spring based, electro-mechanical based, magnetic driver based, and nanomuscle based.

12. The method of claim 1, wherein step (b) is performed with controlled velocity and depth of penetration.

13. The method of claim 1 further comprising:
returning the device to a storage condition without having to dispose of a used penetrating member or used analyte detecting members.

14. The method of claim 1 wherein the device is ready for the next lancing event without having to dispose of the used penetrating member or the used analyte detecting member.

15. The method of claim 1 wherein a time from pressing an on button of the device to lancing and measuring the analyte level is no more than 10 seconds.

16. A method of analyte measurement by a user using an analyte measurement device, comprising:
(a) making a decision to test;
(b) presenting a penetrating member and unused analyte detecting member of the analyte measurement device into an active position;
(c) firing the penetrating member to prick the skin and bring a fluid sample to the analyte detecting member;
(d) measuring the analyte level, and
wherein steps (a) through (d) occur in no more than 1 minute.

17. The method of claim 16, wherein steps (a) through (d) occur in no more than 30 seconds.

18. The method of claim 16, wherein steps (a) through (d) occur in no more than 15 seconds.

19. The method of claim 16, wherein steps (a) through (d) occur in no more than 10 seconds.

20. A method of analyte measurement performed by an analyte measurement device, the method comprising:
(a) presenting a penetrating member and unused analyte detecting member of the analyte measurement device into an active position by rotating a disposable device to align in the active position, removing seals covering the penetrating member and analyte detecting member;
(b) firing the penetrating member to prick the skin using a driver to advance and retract from the skin to create a wound from which body fluid expresses;
(c) bring a fluid sample to the analyte detecting member by providing a sample capture structure positioned to contact body fluid expressed from the wound;
(d) measuring the analyte levels; and wherein steps (a) through (d) are completed in no more than 10 seconds.

21. The method of claim 20, further comprising:
displaying the analyte level to the user

22. The method of claim 20, further comprising:
storing a value of the analyte level.

23. The method of claim 20, wherein steps (a) through (d) are performed without the user directly handling the penetrating member to obtain a fresh penetrating member or load the penetrating member

24. The method of claim 20, wherein steps (a) through (d) are performed without the user coding the analyte measurement device.

25. The method of claim 20, wherein blood is applied to an analyte detection member during lancing.

26. The method of claim 25, wherein the application of blood to an analyte detection member during lancing occurs without removal and disposal of penetrating members from the analyte measurement device.

27. The method of claim 20, wherein steps (a) through (d) are performed without a separate step of apply blood to a analyte detection member after lancing.

28. The method of claim 20, wherein steps (b) and (c) are performed without milking a wound.

29. The method of claim 20, wherein steps (b) and (c) are performed using at least one of a penetrating member drivers selected from, spring based, electromechanical based, magnetic driver based, and nanomuscle based.

30. The method of claim 20, wherein step (c) is performed with controlled velocity and depth of penetration.

31. The method of claim 20, further comprising:
returning the device to a storage condition without having to dispose of a used penetrating member or used analyte detecting members.

32. The method of claim 20, wherein the device is ready for the next lancing event without having to dispose of the used penetrating member or the used analyte detecting member.

33. The method of claim 20, wherein a time from pressing an on button of the device to lancing and measuring the analyte level is no more than 10 seconds.

34. The method of claim 14 wherein there is no disposal of a used analyte detecting member and a used penetrating member after each lancing step.

35. The method of claim 20, wherein steps (a) through (d) are performed without a disposal or handling of waste step.
